(19) United States
(12) Patent Application Publication
(10) Pub. No.: US 2020/0008658 A1
(43) Pub. Date: Jan. 9, 2020

(54) DISTAL END COVER FOR ENDOSCOPE

(71) Applicant: OLYMPUS CORPORATION, Tokyo (JP)

(72) Inventor: Fumitoshi HAYAKAWA, Tokyo (JP)

(73) Assignee: OLYMPUS CORPORATION, Tokyo (JP)

(21) Appl. No.: 16/572,688

(22) Filed: Sep. 17, 2019

Related U.S. Application Data
(63) Continuation of application No. PCT/JP2018/010281, filed on Mar. 15, 2018.

(30) Foreign Application Priority Data
Mar. 31, 2017 (JP) 2017-071208

(51) Int. Cl. A61B 1/00; B29C 45/00

(52) U.S. Cl. CPC A61B 1/00137; A61B 1/00177; B29L 2031/753; B29C 2045/0027; B29C 45/0025

(57) ABSTRACT

A distal end cover for an endoscope molded by injecting resin into a mold for molding, attached to a distal end member of an insertion portion in the endoscope, a raising base being provided in the distal end member , covering at least a part of the distal end member, and having an opening portion configured to expose a space in which the raising base is housed to an outside, includes a thin portion having one end connected to a periphery portion of the opening portion and extending linearly and two resin injection gate marks disposed in such a manner that the thin portion is located between the two resin injection gate marks, in which the two resin injection gate marks are disposed at positions where the resin injected into a cavity through a gate of the mold forms a weld line along the thin portion.

### CROSS REFERENCE TO RELATED APPLICATION

This application is a continuation application of PCT/JP2018/010281 filed on Mar. 15, 2018 and claims benefit of Japanese Application No. 2017-071208 filed in Japan on Mar. 31, 2017, the entire contents of which are incorporated herein by this reference.

### BACKGROUND OF THE INVENTION

### 1. Field of the Invention

The present invention relates to a distal end cover for an endoscope attached to a distal end member configuring a distal end portion of an endoscope insertion portion, a raising base being provided in the distal end member.

### Background Art

One of medical endoscopes is a lateral-viewing type endoscope (hereinafter, described as an endoscope) or a so-called endoscope for duodenum in which an illumination lens and an objective lens are aligned on a side surface of a distal end side of an insertion portion. In the endoscope, a treatment instrument channel and a raising device are provided.

In the treatment instrument channel, a treatment instrument such as a contrast imaging tube, a basket catheter, and a balloon catheter are inserted. The treatment instrument having passed through the treatment instrument channel is led out to an outside through a distal end opening provided in the distal end member, and a lead-out direction is switched to a desired direction by the raising device.

The raising device is configured mainly by a raising base disposed turnably on the distal end member, a raising-base operation lever provided on an operation portion, and a raising-base operation wire configured to move with an operation of the raising-base operation lever and to cause the raising base to swing in general.

An electrically insulated distal end cover for an endoscope is externally attached to the distal end member. The distal end cover is fixed by an adhesive in order to prevent removal from the distal end member.

The endoscope is cleaned and disinfected after use. When the insertion portion of the endoscope is to be cleaned, it is known that cleaning can be easily performed by removing the distal end cover from the distal end portion so as to expose a distal end port of the treatment instrument channel.

The description of Japanese Patent No. 4855824 discloses a distal end cover which can be removed from a distal end member by breaking the distal end cover through ripping in a thin portion without damaging a soft member configuring the insertion portion and can prevent removal during use.

In the distal end cover for an endoscope described above, a thin portion and a recessed groove which are plastic deformation portion configured to sequentially unlock a locked state by a first locking portion, a second locking portion, and a third locking portion by plastically deforming a finger hook portion at a start point.

The thin portion is provided on a side surface portion between the finger hook portion of the distal end cover for an endoscope and the opening portion. The recessed groove is formed on an inner peripheral surface over an entire circumference from a proximal end portion of the thin portion or a vicinity of the proximal end portion to the side surface portion, a front surface portion, and the side surface portion on an opposite side of the distal end cover for an endoscope.

### SUMMARY OF THE INVENTION

A distal end cover for an endoscope according to an aspect of the present invention is molded by injecting resin into a mold for molding, is attached to a distal end member of an insertion portion in the endoscope, a raising base being provided in the distal end member, covers at least a part of the distal end member, and has an opening portion configured to expose a space in which the raising base is housed to an outside. The distal end cover includes a thin portion having one end connected to a periphery portion of the opening portion and extending linearly and two resin injection gate marks disposed in such a manner that the thin portion is located between the two resin injection gate marks, and the two resin injection gate marks are disposed at positions where the resin injected into a cavity through a gate of the mold forms a weld line along the thin portion.

A distal end cover for an endoscope according to another aspect of the present invention is molded by injecting resin into a mold for molding, is attached to a distal end member of an insertion portion in the endoscope, and covers at least a part of the distal end member. The distal end cover includes a thin portion provided in the distal end cover and extending linearly in a predetermined direction and two resin injection gate marks disposed in such a manner that the thin portion is located between the two resin injection gate marks, and the two resin injection gate marks are disposed at positions where the resin injected into a cavity through a gate of the mold forms a weld line along the thin portion.

### BRIEF DESCRIPTION OF THE DRAWINGS

FIG. 1 is a view illustrating a schematic configuration of a lateral-viewing type endoscope;

FIG. 2 is a perspective view of a distal end portion of an insertion portion;

FIG. 3 is perspective view illustrating a state where a distal end cover and a distal end member are separated;

FIG. 4 is a top view of the distal end portion when seen from an up point toward a down point;

FIG. 5 is a top view of a distal end member;

FIG. 6 is a partial sectional view of the distal end cover when seen from the up point toward the down point;

FIG. 7 is a partial sectional view of the distal end portion when seen from the up point toward the down point;

FIG. 8 is a front view of the distal end portion when seen from a distal end point toward a proximal end point;

FIG. 9 is a Ix-IX sectional view of FIG. 7;

FIG. 10 is a X-X sectional view of FIG. 7;

FIG. 11 is a view illustrating a state where a force is applied by hooking a finger on a finger hook portion;

FIG. 12 is a sectional view of a mold for molding configured to mold the distal end cover;

FIG. 13 is a sectional view illustrating the distal end cover in a state where breakage occurred;

FIG. 14 is a perspective view illustrating the distal end member and the distal end cover in which breakage occurred;

FIG. 15 is a view illustrating a state where breakage is generated in the distal end cover by fingers;

FIG. 16 is a sectional view illustrating a modification of the mold for molding; and

FIG. 17 is a partial sectional view illustrating the distal end cover molded by the mold for molding in the modification.

### DETAILED DESCRIPTION OF THE PREFERRED EMBODIMENT

A preferred embodiment of the present invention will be described below by referring to the drawings. In each of the drawings used for the following description, in order to make a size of each of constituent elements to such a degree recognizable on the drawing, scales are different for each of the constituent elements, and the present invention is not limited only to a quantity of the constituent element, a shape of the constituent element, a ratio of sizes of the constituent elements and a relative positional relationship of each of the constituent elements described in these figures.

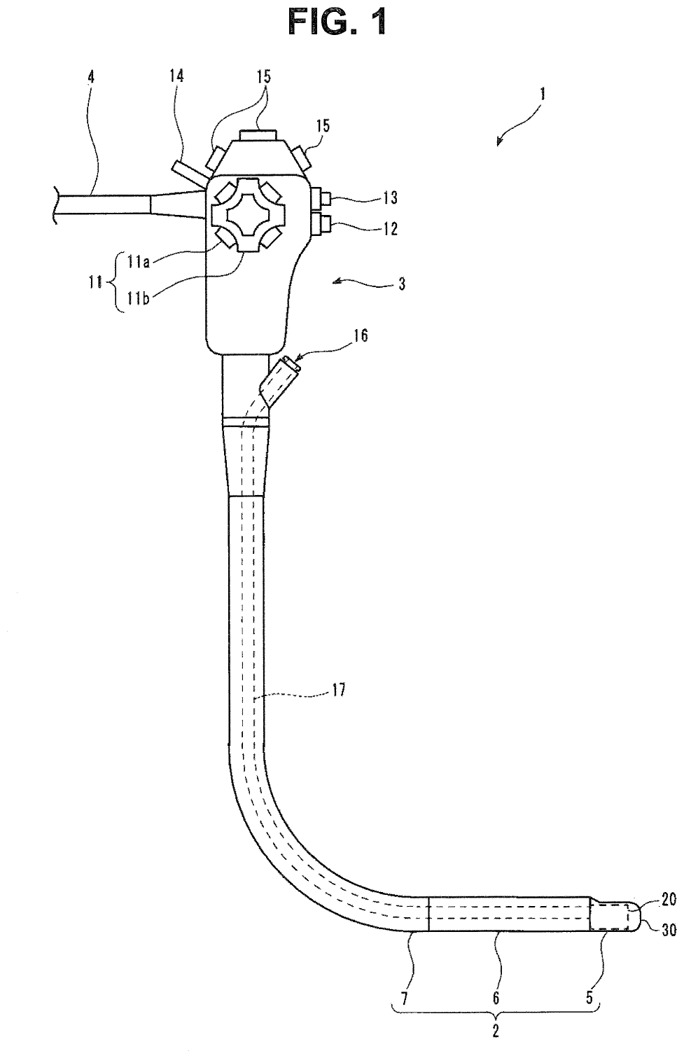

A distal end cover for an endoscope in the embodiment (hereinafter, referred to simply as a distal end cover) is attached to a lateral-viewing type endoscope 1. FIG. 1 is a view illustrating a schematic configuration of the lateral-viewing type endoscope 1 including a distal end cover 30. The distal end cover 30 may be in such a form attached to an endoscope in a format other than the lateral-viewing type.

The endoscope 1 is configured by including an insertion portion 2 configured to be inserted into a subject, an operation portion 3 provided on a proximal end side of the insertion portion 2, and a universal cord 4 extending from the operation portion 3.

A bending operation device 11, an air-feeding/water-feeding button 12, a suction button 13, a raising base operation lever 14, and various operation switches 15 are provided in the operation portion 3.

The operation switches 15 include a freeze switch configured to generate a freeze signal, a release switch configured to generate a release signal when photographing is performed, an observation mode switching switch configured to give a switching instruction of an observation mode.

A treatment instrument insertion port 16 configured to lead a treatment instrument (not shown) into a body is provided in the operation portion 3. One end side of a channel tube 17 is connected to the treatment instrument insertion port 16. The channel tube 17 is inserted into the insertion portion 2. The other end side of the channel tube 17 is connected to a distal end member 20 configuring a distal end portion 5 of the insertion portion 2.

The insertion portion 2 is configured by consecutively providing the distal end portion 5, a bending portion 6, and a flexible tube portion 7 in order from the distal end side. The distal end portion 5 is configured by being attached with the distal end cover 30 for endoscope to the distal end member 20. Details of a constitution of the distal end portion 5 will be described later.

The flexible tube portion 7 is configured by providing a spiral tube, a mesh-state tube covering the spiral tube, and a heat-shrink tube configuring an outermost layer.

The bending portion 6 is configured by providing a bending piece set configured to bend in four directions of up, down, left and right, a mesh-state tube made of metal and covering the bending piece set, and a bending rubber which is a sheath. The bending portion 6 is configured to be bent in an up direction or in a down direction by rotating/operating an up-and-down bending knob 11a of the bending operation device 11 provided in the operation portion 3 and to be bent in a left direction or in a right direction by rotating/operating a left-and-right bending knob 11b.

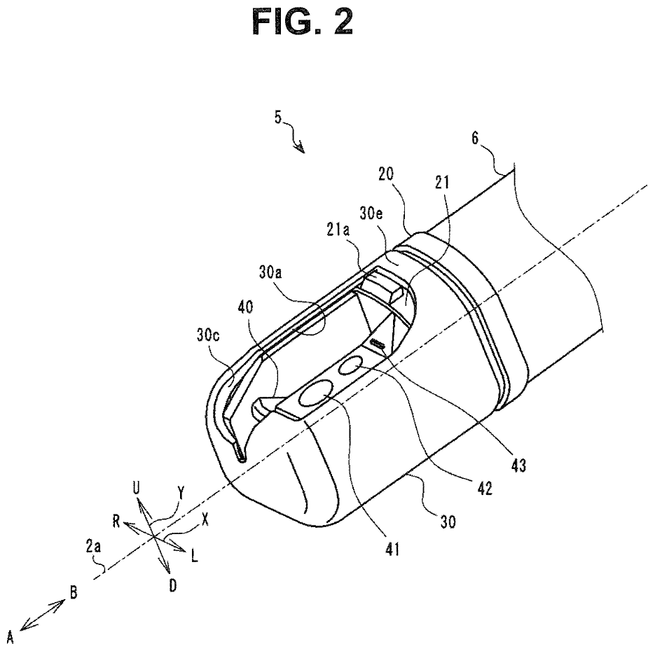

FIG. 2 is a perspective view of the distal end portion 5. As illustrated in FIG. 2, the distal end portion 5 is configured by attaching the distal end cover 30 for endoscope to the distal end member 20. The distal end cover 30 is a sheath-like member covering a predetermined outer surface of the distal end member 20 and can be detachably attached to the distal end member 20. Though details will be described later, the distal end cover 30 has a breakage expected portion provided which is a spot where breakage as irreversible deformation occurs when the distal end cover 30 is removed from the distal end member 20 after once attached to the distal end member 20.

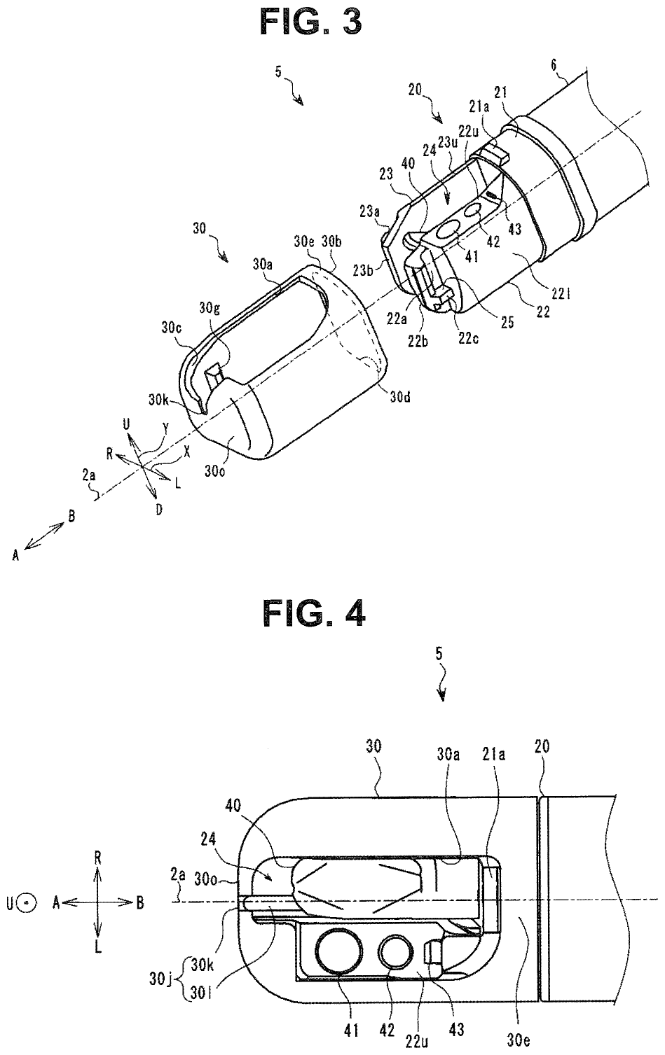

FIG. 3 is a perspective view illustrating the distal end cover 30 and the distal end member 20 in a separated state. In FIG. 3, the distal end cover 30 in a state (unused state) where the distal end cover 30 has never been attached to the distal end member 20 is illustrated.

The distal end member 20 is a rigid member configuring the distal end portion 5, and the distal end cover 30 is configured by resin having electric insulation and has repulsiveness determined in advance. A type of the resin configuring the distal end cover 30 is not particularly limited, but in the embodiment, the distal end cover 30 is formed by resin having electric insulation and elasticity lower than rubber and susceptible to plastic deformation or breakage among resins such as polyethylene and polypropylene as an example. A type of the resin configuring the distal end cover 30 is not particularly limited as long as a condition that the resin has elasticity lower than rubber and is susceptible to plastic deformation or breakage is satisfied. A material configuring a surface of a portion of the distal end member 20 in contact with the distal end cover 30 is more rigid than resin configuring the distal end cover 30. As a result, wear of the distal end member 20 caused by repetition of replacement of the distal end cover 30 can be prevented. The distal end cover 30 is molded by using a mold 60 for molding which will be described later.

The distal end cover 30 is preferably made of translucent or transparent resin and having a color of a material in which a pigment, etc. is not mixed. When the distal end cover 30 is made of translucent or transparent resin, a user of the endoscope 1 can visually recognize easily whether the distal end cover 30 is correctly attached to a predetermined position with respect to the distal end member 20 or not. Moreover, an image contrast substance having transmittance of X-rays largely different from a tissue of a subject such as a human body may be mixed in the resin configuring the distal end cover 30. The image contrast substance may be mixed in the entire distal end cover 30 or may be partially mixed.

In the following description for the configuration of the distal end portion 5, an axis in a longitudinal direction of the elongated insertion portion 2 is called a longitudinal axis 2a. A direction toward a distal end side of the insertion portion 2 along the longitudinal axis 2a is called a distal end direction A, and a direction opposite to the distal end direction A is called a proximal end direction B. Two straight axes orthogonal to each other on a plane orthogonal to the longitudinal axis 2a are defined as an X-axis and a Y-axis. A direction toward one side along the X-axis is called a right direction R, and a direction opposite to the right direction R is called a left direction L. A direction toward one side along the Y-axis is called an up direction U, and a direction opposite to the up direction U is called a down direction D. The X-axis and the Y-axis are substantially in parallel with a bending direction of the bending portion 6.

In the embodiment, an example in which a right side is the right direction R and an upper side is the up direction is assumed in a case when seen from the proximal end side toward the distal end side along the longitudinal axis 2a, and the X axis is horizontal.

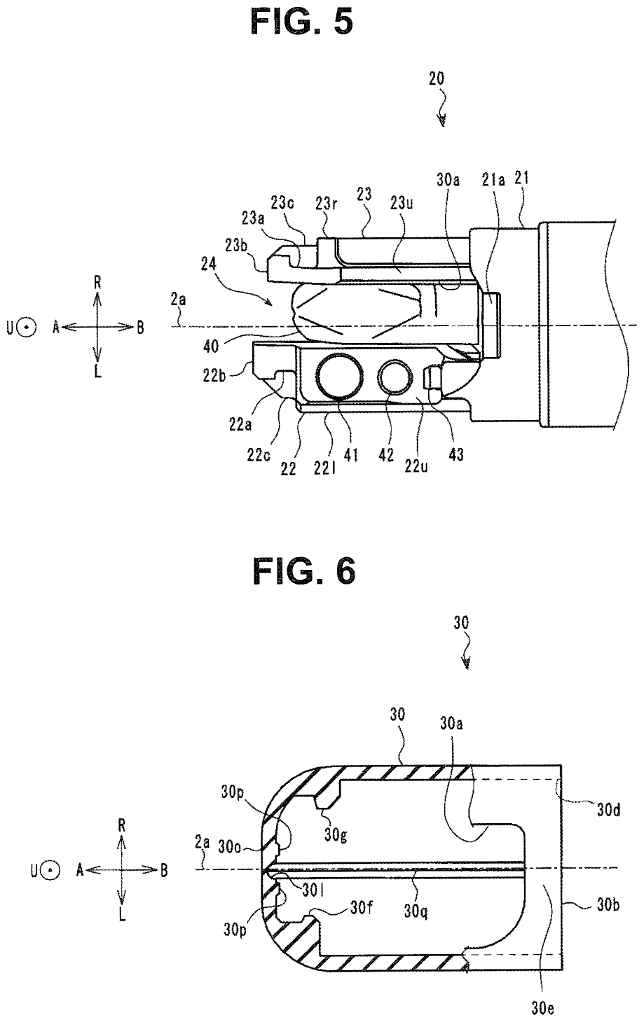

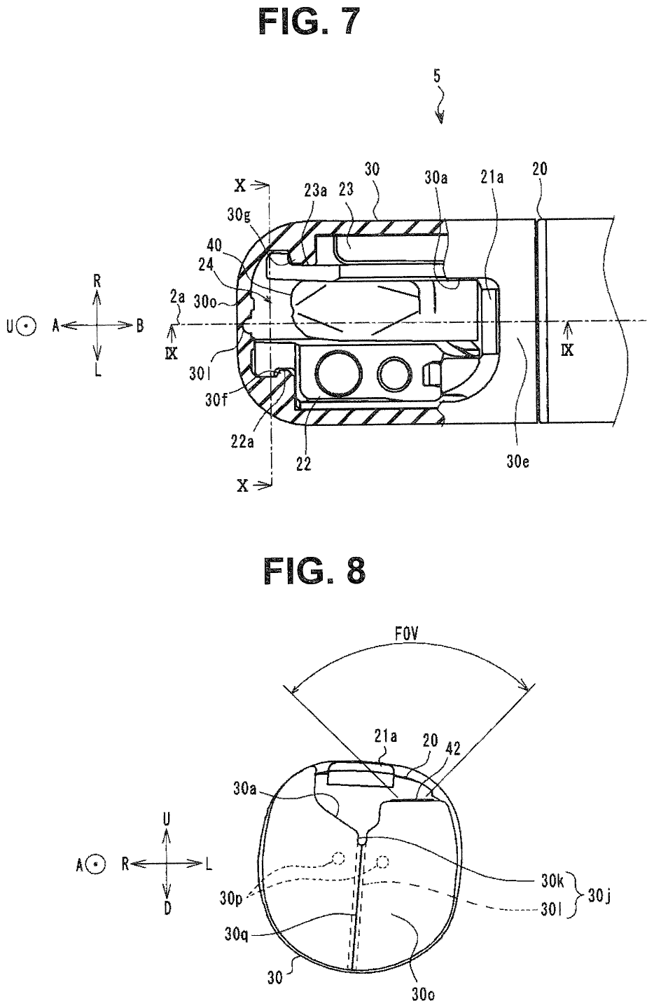

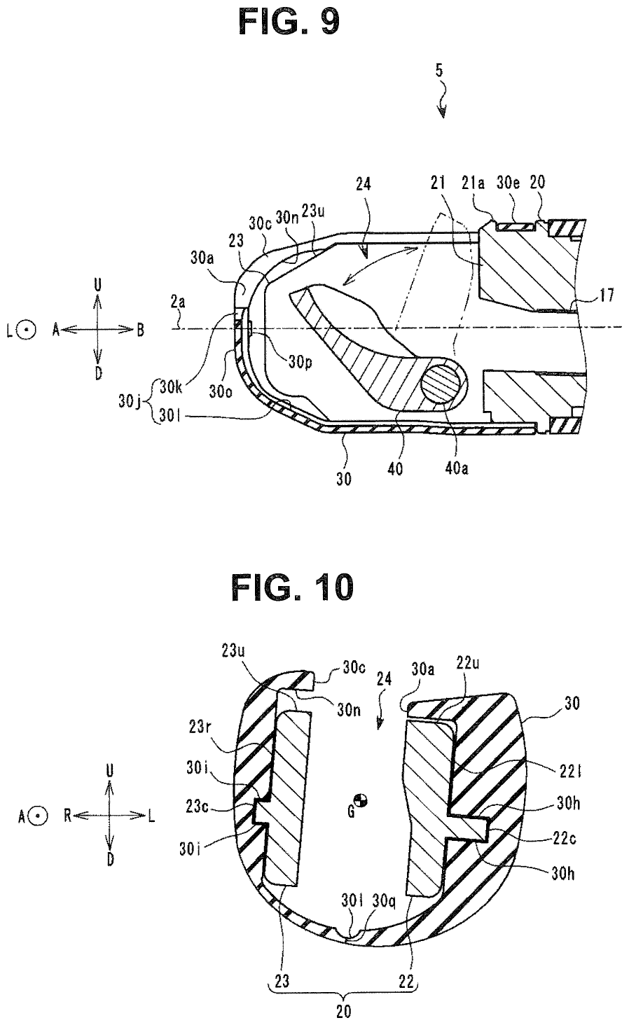

FIG. 4 is a top view of the distal end portion 5 when seen from a point in the up direction U toward a point in the down direction D. FIG. 5 is a top view of the distal end member 20. FIG. 6 is a partial sectional view of the distal end cover 30 when seen from the point in the up direction U toward the point in the down direction D. FIG. 7 is a partial sectional view of the distal end portion 5 when seen from the point in the up direction U toward the point in the down direction D. FIG. 8 is a front view of the distal end portion 5 when seen from a point in the distal end direction A toward a point in the proximal end direction B. FIG. 9 is a IX-IX sectional view of FIG. 7. FIG. 10 is a X-X sectional view of FIG. 7.

As illustrated in FIGS. 3 and 5, the distal end member 20 has a distal end portion body 21, a first arm portion 22 and a second arm portion 23 which are a pair of arm portions protruding from the distal end portion body 21 in the distal end direction A along the longitudinal axis 2a, and a raising base housing space 24 which is a space formed between the first arm portion 22 and the second arm portion 23. A raising base 40 is turnably disposed in the raising base housing space 24.

An outer shape of the distal end portion body 21 is columnar, and a proximal end of the distal end portion body 21 is connected to a distal end of the bending portion 6. A lock claw 21a protruding toward an outside is provided in an area facing the up direction U in an outer peripheral surface of the distal end portion body 21. The lock claw 21a is a portion to engage with an annular portion 30e of the distal end cover 30 which will be described later.

The first arm portion 22 and the second arm portion 23 are disposed so that the raising base housing space 24 which is a space formed between the first arm portion 22 and the second arm portion 23 is open in three directions, that is, in the up direction U, in the down direction D, and in the distal end direction A. In other words, the first arm portion 22 and the second arm portion 23 are aligned in a direction along the X-axis having the raising base housing space 24 between the first arm portion 22 and the second arm portion 23. In the embodiment, the first arm portion 22 is disposed on the left direction L side of the raising base housing space 24, and the second arm portion 23 is disposed on the right direction R side of the raising base housing space 24 as an example.

The first arm portion 22 and the second arm portion 23 are in a cantilever state in the embodiment, and no member is provided to extend between the first arm portion 22 and the second arm portion 23, but a columnar or wall-shaped member connecting the first arm portion 22 and the second arm portion 23 may be provided between the first aim portion 22 and the second arm portion 23.

An illumination lens 41, an observation lens 42, and a cleaning nozzle 43 are disposed on an upper surface 22u facing the up direction U in the outer peripheral surface of the first arm portion 22. The observation lens 42 is for picking up an image of a subject, and the illumination lens 41 is for ejecting illumination light toward the subject. As illustrated in FIG. 8, a field of view FOV of the observation lens 42 has substantially the up direction U as a center. In other words, the observation lens 42 sees a side of the insertion portion 2 in the field of view. The cleaning nozzle 43 is a portion configured to eject a fluid toward the illumination lens 41 and the observation lens 42.

As illustrated in FIG. 9, the raising base 40 is disposed turnably around a turning shaft 40a substantially in parallel with the X-axis in the raising base housing space 24. The raising base 40 is a tongue-shaped member extending in one direction from the turning shaft 40a. FIG. 9 illustrates a turning range of the raising base 40, and the raising base 40 is turned between a first position indicated by a solid line and a second position indicated by a two-dot chain line.

The raising base 40 takes an attitude extending substantially in the distal end direction A from the turning shaft 40a when the raising base 40 is at the first position and the entirety is sandwiched between the first arm portion 22 and the second arm portion 23. When the raising base 40 is at the second position, the raising base 40 takes an attitude extending substantially in the up direction U from the turning shaft 40a, and the distal end portion takes an attitude protruding in the up direction U to be higher than the first arm portion 22 and the second arm portion 23.

A turning operation of the raising base 40 is performed by the raising base operation lever 14 provided in the operation portion 3. A mechanism such as a wire for transmitting movement of the raising base operation lever 14 to the raising base 40 is disposed inside the second arm portion 23.

Returning again to FIGS. 3 and 5 for description, an engagement groove 22a is engraved in a left side surface 22l facing the left direction L in the outer peripheral surface of the first arm portion 22. The engagement groove 22a has a recess shape going from an outer surface of the left side surface 22l toward an inside (right direction R). The engagement groove 22a is provided so as to extend along the Y-axis. In the embodiment, the engagement groove 22a is disposed on a distal end portion 22b of the first arm portion 22 as an example.

An engagement groove 23a is engraved in a right side surface 23r facing the right direction R in the outer peripheral surface of the second arm portion 23. The engagement groove 23a has a recess shape going from an outer surface of the right side surface 23r toward an inside (left direction L). The engagement groove 23a is provided so as to extend along the Y-axis. In the embodiment, the engagement groove 23a is disposed on a distal end portion 23b of the second arm portion 23 as an example.

As illustrated in FIG. 5, the engagement groove 22a of the first arm portion 22 and the engagement groove 23a of the second aim portion 23 are recess-shaped portions open in directions opposite to each other along an axis substantially orthogonal to the longitudinal axis 2a on the outer surface of the distal end member 20. The engagement grooves 22a and 23a formed in the outer surface of the distal end member 20 are portions to engage with lock claws 30f and 30g of the distal end cover 30 which will be described later, respectively.

An abutting surface portion 25 is provided on the outer peripheral surface of the distal end member 20 of the embodiment. The abutting surface portion 25 is configured to restrict relative rotation around the longitudinal axis 2a of the distal end cover 30 with respect to the distal end member 20 in a state where the distal end cover 30 is attached to the distal end member 20.

The abutting surface portion 25 is a planar wall surface portion raised from an area of a surface along the longitudinal axis 2a in the outer peripheral surface of the distal end member 20 and substantially in parallel with the longitudinal axis 2a.

Though details will be described later, when an input is made for relatively rotating the distal end cover 30 with respect to the distal end member 20, a rotation stopping portion 30h configured to abut on the abutting surface portion 25 which is a wall surface portion and to resist the input is provided on an inner peripheral surface of the distal end cover 30. The inner peripheral surface of the distal end cover 30 is a surface faced with the distal end member 20 in a state where the distal end cover 30 is attached to the distal end member 20.

A more specific shape of the abutting surface portion 25 in the embodiment will be described. As illustrated in FIGS. 3 and 10, a plate-shaped first rib 22c standing upright with respect to the left side surface 22l is provided on the left side surface 22l of the first arm portion 22 in the distal end member 20. The first rib 22c is a flat plate-shaped portion along a plane in parallel with the longitudinal axis 2a.

Thus, a surface in the up direction U and a surface in the down direction D of the first rib 22c form a pair of planar wall surface portions raised from the surface (left side surface 22l) along the longitudinal axis 2a in the outer peripheral surface of the distal end member 20 and substantially in parallel with the longitudinal axis 2a. In other words, the surface in the up direction U and the surface in the down direction D of the first rib 22c configure the abutting surface portion 25.

In the embodiment, the first rib 22c is disposed at a position biased to either one of the directions faced with the abutting surface portion 25 which is the wall surface portion with respect to a gravity center position G derived from a profile of the distal end member 20 in a section orthogonal to the longitudinal axis 2a.

More specifically, as illustrated in FIG. 10, the first rib 22c in the embodiment is disposed at a position biased in the down direction D with respect to the gravity center position G derived from the profile of the distal end member 20 in the section orthogonal to the longitudinal axis 2a.

In the embodiment, a plate-shaped second rib 23c standing upright with respect to the right side surface 23r is provided also on the right side surface 23r of the second arm portion 23 in the distal end member 20 as an example.

Next, detailed configuration of the distal end cover 30 attached to the distal end member 20 described above will be described.

As illustrated in FIGS. 2, 4, and 6, the distal end cover 30 is a sheath-like member in which the distal end direction A side is closed and the proximal end direction B side is open and covers a predetermined portion of the outer peripheral surface of the distal end member 20 in a state attached to the distal end member 20.

An opening provided on the proximal end direction B side of the distal end cover 30 is termed an insertion port 30d. When the distal end cover 30 is attached to the distal end member 20, the distal end member 20 is inserted into the distal end cover 30 through the insertion port 30d.

The distal end cover 30 has an opening portion 30a configured to expose the raising base housing space 24, the illumination lens 41, and the observation lens 42 only in the up direction U in the state attached to the distal end member 20. In the state where the distal end cover 30 is attached to the distal end member 20, the illumination lens 41, the observation lens 42, and the cleaning nozzle 43 are also exposed in the up direction U through the opening portion 30a. An R portion 30m having a predetermined diameter is provided on an inner wall surface in the proximal end direction B of the observation lens 42 in the opening portion 30a.

The opening portion 30a is a through hole penetrating a part of the upper surface faced in the up direction U of the distal end cover 30. The opening portion 30a is not in contact with a proximal end 30b which is an end in the proximal end direction B of the distal end cover 30. In other words, the opening portion 30a is not connected to the insertion port 30d on the outer surface of the distal end cover 30. Thus, the proximal end 30b of the distal end cover 30 has the annular portion 30e in which an entire circumference is annularly connected fanned around the longitudinal axis 2a.

In the state where the distal end cover 30 is attached to the distal end member 20, the annular portion 30e is in close contact with an outer peripheral surface of the distal end portion body 21 closer to the proximal end direction B side than the lock claw 21a provided on the distal end portion body 21. In this state, the lock claw 21a provided on the distal end portion body 21 protrudes into the opening portion 30a. In other words, in the state where the distal end cover 30 is attached to the distal end member 20, the lock claw 21a engages with the annular portion 30e and restricts relative movement of the distal end cover 30 in the distal end direction A with respect to the distal end member 20. In this state, the lock claw 21a is provided at a position not in contact with the R portion 30m described above.

Though not shown, an area along the longitudinal axis 2a in the periphery portion of the opening portion 30a is preferably formed thick in the embodiment. Since the thick portion is formed on the periphery portion of the opening portion 30a, deformation of the opening portion 30a when the distal end member 20 is relatively pushed into the distal end cover 30 along the longitudinal axis 2a is prevented, the lock claw 21a can easily pass through the annular portion 30e.

The distal end cover 30 has the lock claws 30f and 30g, the rotation stopping portion 30h, the finger hook portion 30c, and a breakage guiding portion 30j.

As illustrated in FIGS. 6 and 7, the lock claws 30f and 30g are projection-shaped portions protruding from the inner peripheral surface of the distal end cover 30 toward the inside.

The lock claw 30f protrudes in the right direction R from an area facing the right direction R in the inner peripheral surface of the distal end cover 30. The lock claw 30f is engaged/disposed in the engagement groove 22a provided on the left side surface 22l of the first arm portion 22 in the state where the distal end cover 30 is attached to the distal end member 20.

The lock claw 30g protrudes in the left direction L from an area facing the left direction L in the inner peripheral surface of the distal end cover 30. The lock claw 30g is engaged/disposed in the engagement groove 23a provided on the right side surface 23r of the second arm portion 23 in the state where the distal end cover 30 is attached to the distal end member 20.

As described above, the engagement groove 22a of the first arm portion 22 and the engagement groove 23a of the second arm portion 23 form a pair of recess portions open in the directions opposite to each other along the axis substantially orthogonal to the longitudinal axis 2a on the outer surface in a vicinity of the distal end of the distal end member 20. Therefore, relative movement of the distal end cover 30 in the distal end direction A with respect to the distal end member 20 is restricted by engaging the lock claws 30f and 30g of the distal end cover 30 in the engagement grooves 22a and 23a, in the state where the distal end cover 30 is attached to the distal end member 20. The engagement of the lock claws 30f and 30g in the engagement grooves 22a and 23a can be visually recognized by forming the distal end cover 30 with a transparent or translucent resin. The engagement can also be visually recognized not by forming the entire distal end cover 30 with the transparent or translucent resin but by forming only a portion where the lock claws 30f and 30g are provided with the transparent or translucent resin.

As illustrated in FIG. 10, the rotation stopping portion 30h is provided on the inner peripheral surface of the distal end cover 30. The rotation stopping portion 30h is configured to restrict relative rotation of the distal end cover 30 around the longitudinal axis 2a with respect to the distal end member 20 in the state where the distal end cover 30 is attached to the distal end member 20.

When an input is made for relatively rotating the distal end cover 30 around the longitudinal axis 2a with respect to the distal end member 20 in the state where the distal end cover 30 is attached to the distal end member 20, the rotation stopping portion 30h abuts on the abutting surface portion 25 provided on the distal end member 20 and resists the input.

More specifically, the rotation stopping portion 30h is a pair of wall surface portions disposed so as to sandwich the first rib 22c provided on the distal end member 20 from above in the up direction U and below in the down direction D. As described above, the abutting surface portion 25 of the embodiment is configured by including a surface in the up direction U and a surface in the down direction D of the first rib 22c. The rotation stopping portion 30h of the embodiment is configured by a portion faced with the surface in the up direction U and the surface in the down direction D of the first rib 22c in the state where the distal end cover 30 is attached to the distal end member 20. The abutting surface portion 25 and the rotation stopping portion 30h may be in contact or may be separated in the state where the distal end cover 30 is attached to the distal end member 20.

As described above, the first rib 22c is disposed at the position biased in the down direction D with respect to the gravity center position G of the distal end member 20. Thus, when the distal end cover 30 is to be attached to the distal end member 20 at a wrong angle around the longitudinal axis 2a, since the first rib 22c is not inserted into a space between the pair of abutting surface portions 25, attachment cannot be accomplished.

In the embodiment, the distal end cover 30 also includes a rotation stopping portion 30i made of a pair of wall surface portions disposed so as to sandwich the second rib 23c provided on the distal end member 20 from above in the up direction U and below in the down direction D. The rotation of the distal end cover 30 with respect to the distal end member 20 during use of the endoscope 1 can be reliably restricted by providing the rotation stopping portions at two spots as in the embodiment. In the embodiment, as illustrated in FIG. 10, the positions in the up direction U (or the down direction D) of the first rib 22c and the second rib 23c with respect to the gravity center position G are substantially the same, but the positions in the up direction U (or the down direction D) of the first rib 22c and the second rib 23c with respect to the gravity center position G may be different. By making the positions in the up direction U (or the down direction D) of the first rib 22c and the second rib 23c with respect to the gravity center position G different depending on a type of a product, wrong attachment of the distal end cover 30 to the product of the different type can be prevented.

As illustrated in FIGS. 9 and 10, the finger hook portion 30c is provided in the area along the longitudinal axis 2a in the periphery portion of the opening portion 30a of the distal end cover 30 and is a portion where a force is input for relatively rotating the distal end cover 30 around the longitudinal axis 2a with respect to the distal end member 20 by a user's finger or the like in the state where the distal end cover 30 is attached to the distal end member 20.

The areas along the longitudinal axis 2a in the periphery portion of the opening portion 30a are two spots, that is, an area disposed on the first arm portion 22 of the distal end member 20 and an area disposed on the second arm portion 23 in the state where the distal end cover 30 is attached to the distal end member 20. In the embodiment, the finger hook portion 30c is provided on the area disposed on the second arm portion 23 in the periphery portion of the opening portion 30a as an example.

The finger hook portion 30c protrudes in the up direction U to be higher than an upper surface 23u of the second arm portion 23 in the section orthogonal to the longitudinal axis 2a. The finger hook portion 30c protrudes in the up direction U to be higher than the area disposed on the first arm portion 22 in the periphery portion of the opening portion 30a.

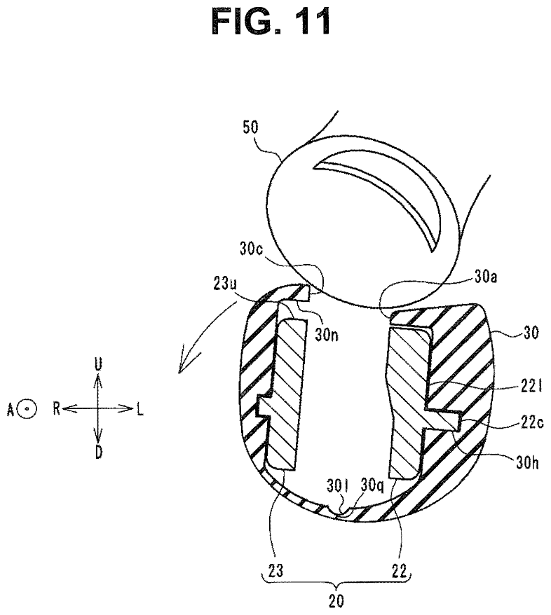

Since the finger hook portion 30c protruding in the up direction U is provided in the periphery portion of the opening portion 30a, an input operation into the distal end cover 30 to be rotated around the longitudinal axis 2a by a user's finger 50 is made easy as illustrated in FIG. 11. FIG. 11 illustrates the user's finger 50 in a state not covered by a glove, but fingers of a person who actually operates the distal end portion 5 of the endoscope 1 after use are in a state covered by a glove.

A gap 30n is formed between the finger hook portion 30c and the upper surface 23u of the second arm portion 23. The input of the user's finger 50 into the finger hook portion 30c is made easier by the gap 30n.

In the embodiment, since the finger hook portion 30c protruding in the up direction U is disposed at a position with the opening portion 30a between the finger hook portion 30c and the observation lens 42, the finger hook portion 30c is disposed at a position relatively far from the observation lens 42. Thus, in the embodiment, the finger hook portion 30c does not enter the field of view FOV of the observation lens 42 or narrow an observation range by the observation lens 42. In other words, in the embodiment, a protrusion amount of the finger hook portion 30c in the up direction U can be made higher within a range not entering the field of view FOV of the observation lens 42, and the input into the finger hook portion 30c by the user's finger 50 can be made easier.

As illustrated in FIGS. 6, 8, and 9, the breakage guiding portion 30j is made of a notch portion 30k and a thin portion 30l. A position of the notch portion 30k in an up-and-down direction (UD direction) in FIG. 8 is set so as to be located in the down direction D rather than an upper end portion in the U direction of the raising base 40 in a state where a distal end portion of the raising base 40 is moved in the down direction D to the farthest.

The notch portion 30k is formed in an end portion on the distal end direction A side in the periphery portion of the opening portion 30a. The end portion on the distal end direction A side in the periphery portion of the opening portion 30a is located on a front surface 30o facing the distal end direction A side of the distal end cover 30.

The notch portion 30k has a shape obtained by notching a periphery portion of the opening portion 30a in the front surface 30o of the distal end cover 30 in a direction substantially orthogonal to the periphery portion. In the embodiment, the notch portion 30k is a portion notched into a substantially V-shape for a predetermined length toward the front surface 30o and in the down direction D from the periphery portion of the opening portion 30a in the front surface 30o of the distal end cover 30. The notched shape of the notch portion 30k may be a U-shape or a rectangular shape.

The thin portion 30l is a linear thin portion extending from the front surface 30o of the distal end cover 30 to a vicinity of the proximal end 30b. Here, the thin portion is a portion in which a thickness is smaller than a thickness in the other portions of the distal end cover 30 and strength to tear is smaller than strength to tear in the other portions.

The thin portion 30l of the embodiment is formed by providing a groove in the inner peripheral surface of the distal end cover 30. The thin portion 30l extends in the down direction D from the notch portion 30k on the front surface 30o of the distal end cover 30 and extends linearly in the proximal end direction B substantially in parallel with the longitudinal axis 2a on the side surface portion of the distal end cover 30. The thin portion 30l is disposed so as to pass between the pair of lock claws 30f and 30g provided on the inner peripheral surface of the distal end cover 30.

As illustrated in FIG. 9, the front surface 30o and a lower surface 30q of the distal end cover 30 are connected by a taper portion 30r. Here, the lower surface 30q is a surface directed in the down direction D on the side surface of the distal end cover 30. The taper portion 30r has an outer shape getting closer to the insertion portion longitudinal axis 2a as the taper portion 30r goes in the distal end direction A. The thin portion 30l is provided along the front surface 30o, the taper portion 30r, and the lower surface 30q. Since the taper portion 30r is provided, breakage generated in the thin portion 30l of the front surface 30o starting at the notch portion 30k can be extended in the proximal end direction B up to the thin portion 30l of the lower surface 30q more easily.

As illustrated in FIG. 9, in the embodiment, when the raising base 40 takes an attitude extending in the distal end direction A from the turning shaft 40a, and the distal end portion of the raising base 40 is moved in the down direction D side to the farthest, the notch portion 30k is located closer to the down direction D side than the distal end portion of the raising base 40. Thus, in the embodiment, the treatment instrument protruding along the raising base 40 is not brought into contact with the notch portion 30k, and occurrence of breakage in the notch portion 30k caused by contact with the treatment instrument is prevented.

In the distal end cover 30 of the embodiment, as illustrated in FIGS. 6, 8, and 9, two resin injection gate marks which are marks of gates for injecting resin (hereinafter, called simply as two gate marks) 30p are disposed in such a manner that the thin portion 30l of the breakage guiding portion 30j is located between two resin injection gate marks.

The two gate marks 30p are marks generated at spots obtained by cutting a runner portion out of the distal end cover 30 in a process of molding the distal end cover 30 by using the mold 60 for molding. The two gate marks 30p are generated at positions of two resin injection gates 62 which are spots where the resin flows into a cavity 61 of the mold 60 for molding. Since the gate mark in an injection molded product made of resin such as the distal end cover 30 is a generally used term for a person ordinally skilled in the art, detailed description will be omitted.

The two gate marks 30p are illustrated as each having a projecting shape for easy discrimination in the figure, but the shapes of the two gate marks 30p are not particularly limited. The shapes of the two gate marks 30p can be different shapes depending on a difference in a method of cutting a runner, presence of finishing after cutting of the runner. Usually, each of the two gate marks 30p has a shape with a spot where the runner is cut protruding to the other surfaces of the distal end cover 30, but each of the spots of the two gate marks 30p where the runner is cut may have a shape recessed toward the other surfaces of the distal end cover 30. Alternatively, each of the two gate marks 30p may have a shape with the spot where the runner is cut flat to the other surfaces of the distal end cover 30.

The two gate marks 30p may be provided on the inner peripheral surface of the distal end cover 30 or may be provided on the outer peripheral surface of the distal end cover 30. In the embodiment, the two gate marks 30p are provided on the inner peripheral surface of the distal end cover 30 as an example. The inner peripheral surface of the distal end cover 30 is a surface faced with the distal end member 20 in the state where the distal end cover 30 is attached to the distal end member 20.

In the embodiment, the two gate marks 30p are disposed on the surface faced with the distal end which is an end on the distal end direction A side of the distal end member 20 in the inner peripheral surface of the distal end cover 30 as an example. In other words, the two gate marks 30p are disposed on a rear side of the front surface 30o of the distal end cover 30. The inner peripheral surface of the distal end cover 30 and the distal end surface of the distal end member 20 are preferably in close contact. However, there can be a case for reasons in a manufacturing process in which the two gate marks 30p largely protrude to the distal end cover 30 side, the two gate marks 30p abut on the distal end surface of the distal end member 20 and undermine the close contact. In order to prevent such a situation, a recess portion in which the two gate marks 30p are contained only needs to be provided in advance in the distal end surface of the distal end member 20 facing the two gate marks 30p.

FIG. 12 is a sectional view of the mold 60 for molding that molds the distal end cover 30. The mold 60 for molding is configured by combining a plurality of molds. FIG. 10 illustrates a state in which the cavity 61 configured to mold the distal end cover 30 is formed by combining the molds 60 for molding. In the state in FIG. 10, the distal end cover 30 is molded by injecting the resin into the cavity 61 through the resin injection gate 62. FIG. 12 illustrates a section by a plane in parallel with the X-axis of the distal end cover 30 molded in the cavity 61 and the longitudinal axis of the insertion portion.

In the embodiment, the mold 60 for molding has an outer die 60a having a hole-shaped recess portion forming the outer peripheral surface of the distal end cover 30 and a pin-shaped inner die 60b inserted into the recess portion of the outer die 60a as an example. As illustrated in FIG. 12, in a state where the inner die 60b is inserted into the recess portion of the outer die 60a, the cavity 61 is formed between the outer die 60a and the inner die 60b. In other words, the outer peripheral surface of the inner die 60b has a shape that forms the inner peripheral surface of the distal end cover 30. The outer die 60a may have a form divided into two parts by a plane in parallel with the drawing of FIG. 12. The inner die 60b may have a form divided into two parts by a plane in parallel with the drawing in FIG. 12.

A linear projecting portion 64 formed so as to form the thin portion 30l described above is provided on the inner die 60b having a shape that forms the inner peripheral surface of the distal end cover 30. In the embodiment, the two resin injection gates 62 and a runner 63 for injecting the resin into the cavity 61 are provided in the inner die 60b as an example. In other words, in the mold 60 for molding in the embodiment, the two resin injection gates 62 are provided in the surface where the linear projecting portion 64 formed so as to form the thin portion 30l is provided. The runner 63 may be provided in plurality.

The two resin injection gates 62 are aligned in a direction crossing a direction in which the linear projecting portion 64 extends on the outer peripheral surface on which the linear projecting portion 64 of the inner die 60b is formed. In other words, the two resin injection gates 62 are disposed so as to sandwich the linear projecting portion 64 on the outer peripheral surface on which the linear projecting portion 64 of the inner die 60b is formed. The two resin injection gates 62 are disposed at positions where a weld line 30q is generated along the linear projecting portion 64. Since the weld line is a well-known term, detailed description will be omitted, but the weld line is a linear area generated at a spot where two resin flows flowing into the cavity 61 from the two resin injection gates 62 merge.

More specifically, the two resin injection gates 62 are disposed line-symmetrically to the linear projecting portion 64 on the outer peripheral surface of the inner die 60b. As described above, distances from the two resin injection gates 62 to the linear projecting portion 64 become equal by disposing the two resin injection gates 62 line-symmetrically to the linear projecting portion 64 and thus, the weld line 30q is generated along the linear projecting portion 64.

In other words, in the distal end cover 30 molded by the mold 60 for molding in the embodiment, as illustrated in FIGS. 6 and 8, the two gate marks 30p are disposed line-symmetrically to the thin portion 30l. In the distal end cover 30 molded by the mold 60 for molding in the embodiment, the weld line 30q is formed along the thin portion 30l. The weld line 30q is not limited to a line appearing with a shape such as a dent generated in the surface of the distal end cover 30, and the weld line 30q cannot be visually recognized clearly as illustrated depending on a material of the resin in many cases.

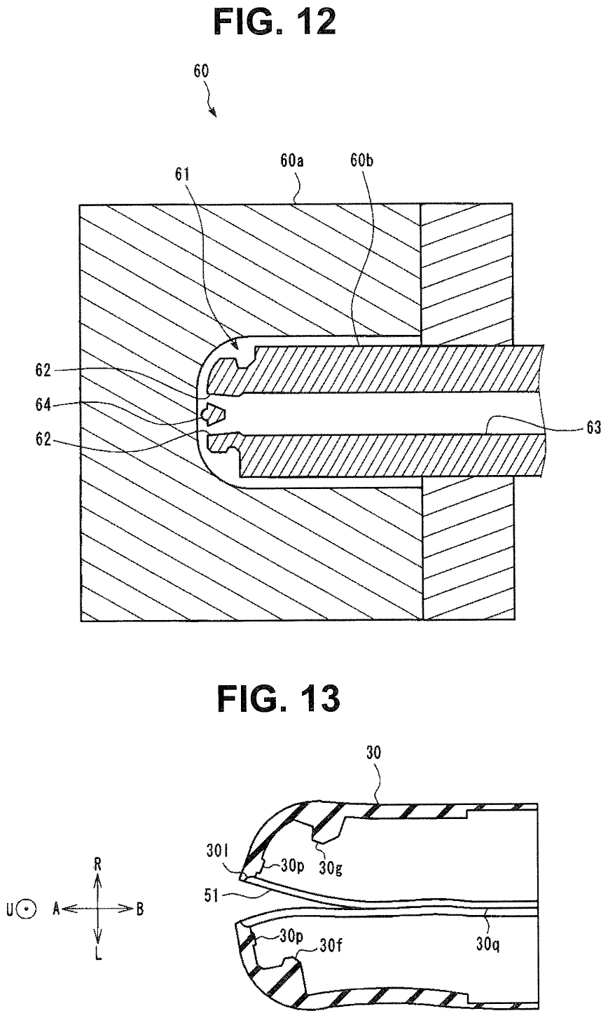

Since the breakage guiding portion 30j is formed in the distal end cover 30 in the embodiment described above, as illustrated in FIG. 13, when the force in a direction in which an opening width of the opening portion 30a is opened in the X-axis direction is input, a breakage 51 along the thin portion 30l is generated in the distal end cover 30 at the notch portion 30k as a start point.

In the state where the distal end cover 30 is attached to the distal end member 20, if the breakage 51 occurs, the force restraining a distance between the pair of lock claws 30f and 30g provided on the inner peripheral surface of the distal end cover 30 is weakened. Thus, as illustrated in FIG. 14, in the distal end cover 30 in which the breakage 51 occurred, engagement between the lock claws 30f and 30g and the engagement grooves 22a and 23a provided in the distal end member 20 is made impossible. Thus, if the breakage 51 occurs in the state where the distal end cover 30 is attached to the distal end member 20, the distal end cover 30 can be removed from the distal end member 20 easily. The distal end cover 30 in which the breakage 51 occurred cannot be fixed to the distal end member 20 anymore.

In order to generate the breakage 51 in the distal end cover 30, the force in a direction that opens the opening width of the opening portion 30a in the X-axis direction needs to be input into the distal end cover 30, but in the embodiment, the force in the direction that opens the opening width of the opening portion 30a in the X-axis direction can be easily input by applying a force that rotates the distal end cover 30 around the longitudinal axis 2a by placing the user's finger 50 on the finger hook portion 30c provided on the periphery portion of the opening portion 30a, as illustrated in FIG. 11.

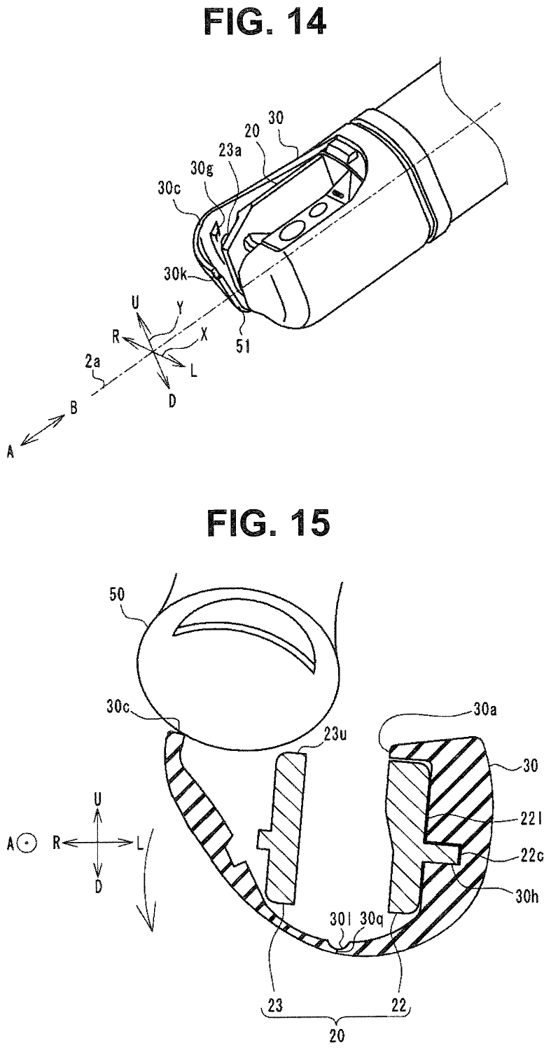

In the embodiment, since the distal end cover 30 includes the rotation stopping portion 30h configured to restrict rotation with respect to the distal end member 20, there is no need for a user to input the force that restricts the rotation of the distal end cover 30 when the breakage 51 is to be generated in the distal end cover 30. Therefore, according to the embodiment, the user can generate the breakage 51 in the distal end cover 30, while the distal end portion 5 is held by one hand, only by applying a force in one direction to the finger hook portion 30c by the finger 50 of the other hand as illustrated in FIG. 15. Since the force applied to the distal end cover 30 is in one direction, the force can be input easily even with the finger covered by a glove. When the finger hook portion 30c is picked by the fingers and the force is further applied in an arrow direction from the state illustrated in FIG. 14, the distal end cover 30 is rotated in a counterclockwise direction in FIG. 14, the R portion 30m of the distal end cover 30 abuts on the one end of the lock claw 21a and then, the R portion 30m goes over the lock claw 21a. At this time, a diameter of the annular portion 30e is somewhat enlarged by the lock claw 21a. By means of the rotation of the distal end cover 30, the lock claw 21a sneaks into an inside of the annular portion 30e. In the state where the lock claw 21a sneaks into the inside of the annular portion 30e, the distal end cover 30 can be removed easily from the distal end member 20 with a weak force by pulling the finger hook portion 30c to the distal end direction A side.

In the embodiment, the weld line 30q is formed along the thin portion 30l. In general, strength of a spot where a weld line is formed in resin molded product is lowered. In other words, in the embodiment, since the strength of the thin portion 30l can be lowered by generation of the weld line 30q, the breakage 51 can be generated in the thin portion 30l with a relatively weak force.

As described above, the distal end cover 30 in the embodiment can be easily removed from the distal end member 20 with the finger covered by a glove, and removal from the distal end member 20 during use can be prevented.

In the embodiment, the rotation stopping portion 30h is disposed on a side opposite to the finger hook portion 30c which is an input point of the force by sandwiching the gravity center position G derived from the profile of the distal end member 20 in the section orthogonal to the longitudinal axis 2a. In other words, when the distal end cover 30 is seen from a line along the longitudinal axis 2a, the finger hook portion 30c is disposed on the side opposite to the rotation stopping portion 30h by sandwiching the opening portion 30a and the breakage guiding portion 30j. Therefore, after the input to the finger hook portion 30c is started by the finger 50, first, a portion located at the farthest from the rotation stopping portion 30h of the distal end cover 30 is separated from the distal end member 20. Therefore, at a stage where the breakage 51 begins to occur in the distal end cover 30, a state where the rotation stopping portion 30h abuts on the abutting surface portion 25 can be reliably maintained.

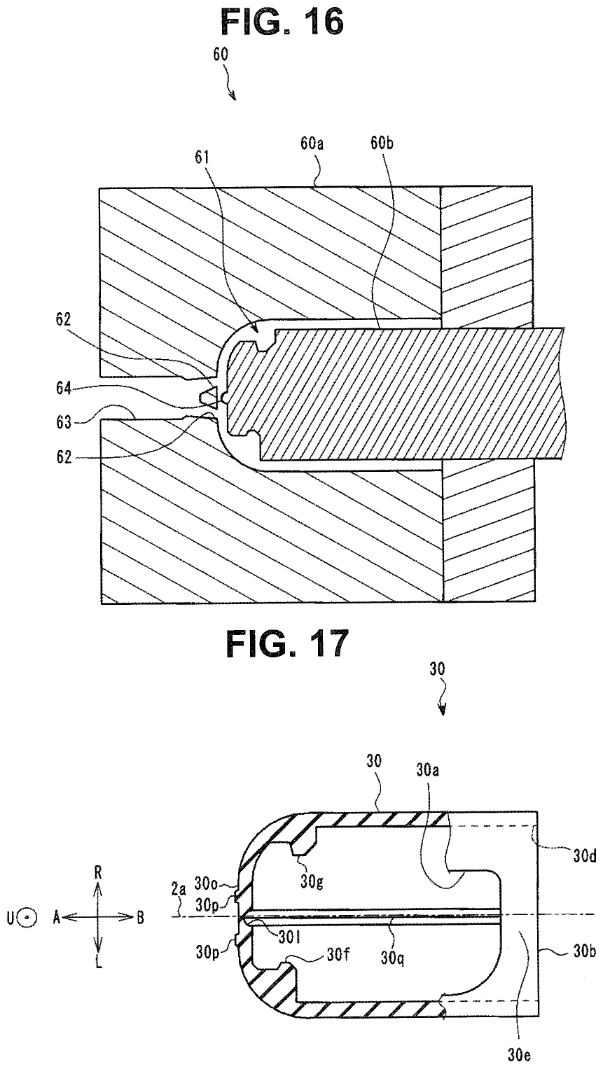

In the embodiment described above, the two resin injection gates 62 and the runner 63 are provided in the inner die 60b of the mold 60 for molding, but the two resin injection gates 62 and the runner 63 may be provided in the outer die 60a as in a variation of the mold 60 for molding illustrated in FIG. 16. In the variation, the runner 63 may be in plural.

In the variation illustrated in FIG. 16, similarly to the embodiment described above, the two resin injection gates 62 are disposed in the surface facing the linear projecting portion 64 in the cavity 61. The two resin injection gates 62 are aligned in the direction crossing the direction in which the linear projecting portion 64 extends and are disposed at positions where the weld line 30q is generated along the linear projecting portion 64.

Therefore, in the distal end cover 30 molded by the mold 60 for molding in the variation, as illustrated in FIG. 17, the two gate marks 30p are disposed in such a manner that the thin portion 30l is located between the two gate marks 30p on the outer peripheral surface of the distal end cover 30. In more detail, the two gate marks 30p are disposed line-symmetrically to the thin portion 30l. In the distal end cover 30 molded by the mold 60 for molding in the variation, the weld line 30q is formed along the thin portion 39l.

Therefore, in the distal end cover 30 in the variation, too, the strength of the thin portion 30l can be lowered by forming the weld line 30q in the thin portion 30l, and the breakage 51 can be generated in the thin portion 30l with a relatively weak force.

The present invention is not limited to the embodiment described above but can be changed as appropriate within a range not contradicting a gist or an idea of the invention that can be read out of the claims and the entire description, and the distal end cover for an endoscope involving such changes is also included in the technical range of the present invention.

What is claimed is:

1. A distal end cover for an endoscope, the distal end cover being molded by injecting resin into a mold for molding, being attached to a distal end member of an insertion portion in the endoscope, a raising base being provided in the distal end member, covering at least a part of the distal end member, and having an opening portion configured to expose a space in which the raising base is housed to an outside, the distal end cover comprising:
a thin portion having one end connected to a periphery portion of the opening portion and extending linearly; and
two resin injection gate marks disposed in such a manner that the thin portion is located between the two resin injection gate marks, wherein
the two resin injection gate marks are disposed at positions where the resin injected into a cavity through a gate of the mold forms a weld line along the thin portion.

2. The distal end cover for an endoscope according to claim 1, wherein
the two resin injection gate marks are disposed line-symmetrically to the thin portion.

3. The distal end cover for an endoscope according to claim 1, wherein
the two resin injection gate marks are disposed on a surface faced with the distal end member.

4. The distal end cover for an endoscope according to claim 1, wherein
the thin portion includes a recess portion in a surface faced with the distal end member.

5. The distal end cover for an endoscope according to claim 1, wherein
the thin portion is provided on a surface constituting a surface directed in an insertion direction in the insertion portion.

6. The distal end cover for an endoscope according to claim 5, wherein
another end of the thin portion extends to a surface adjacent to a side of the surface directed in the insertion direction.

7. The distal end cover for an endoscope according to claim 5, wherein
the thin portion extends from the opening portion to a surface faced with the opening portion.

8. The distal end cover for an endoscope according to claim 5, wherein
the thin portion extends to a rear end of the distal end cover.

9. A distal end cover for an endoscope, the distal end cover being molded by injecting resin into a mold for molding, being attached to a distal end member of an insertion portion in the endoscope, and covering at least a part of the distal end member, the distal end cover comprising:
a thin portion provided on the distal end cover and extending linearly in a predetermined direction; and
two resin injection gate marks disposed in such a manner that the thin portion is located between the two resin injection gate marks, wherein
the two resin injection gate marks are disposed at positions where the resin injected into a cavity through a gate of the mold forms a weld line along the thin portion.

10. The distal end cover for an endoscope according to claim 9, wherein
the two resin injection gate marks are disposed line-symmetrically to the thin portion.

11. The distal end cover for an endoscope according to claim 9, wherein
the two resin injection gate marks are disposed on a surface side faced with the distal end member.

12. The distal end cover for an endoscope according to claim 9, wherein
the thin portion includes a recess portion in a surface faced with the distal end member.
